(19) United States
(12) Patent Application Publication
(10) Pub. No.: US 2014/0086995 A1
(43) Pub. Date: Mar. 27, 2014

(54) POLYMER MICROSPHERE COMPOSITIONS FOR LOCALIZED DELIVERY OF THERAPEUTIC AGENTS

(71) Applicants: Buddy D. Ratner, Seattle, WA (US); Julee Floyd, Seattle, WA (US); Rohan Ramakrishna, Seattle, WA (US); Anna Galperin, Seattle, WA (US)

(72) Inventors: Buddy D. Ratner, Seattle, WA (US); Julee Floyd, Seattle, WA (US); Rohan Ramakrishna, Seattle, WA (US); Anna Galperin, Seattle, WA (US)

(21) Appl. No.: 14/111,193

(22) PCT Filed: Apr. 12, 2012
(86) PCT No.: PCT/US2012/033383

§ 371 Date: Nov. 26, 2013

Related U.S. Application Data
(60) Provisional application No. 61/474,631, filed on Apr. 12, 2011.

(51) Int. Cl. A61K 9/50; A61K 31/5377

(52) U.S. Cl. CPC A61K 9/50; A61K 31/5377
USPC 424/489; 514/234.5

(57) ABSTRACT

Compositions and methods for localized delivery of a therapeutic agent to a biological tissue over time. The composition includes a temperature-responsive polymer and one or more microspheres, each having degradation rate different from the other, and each comprising a therapeutic agent. In the method, the composition is applied to a biological tissue and forms a gel that adheres to the tissue.

### CROSS-REFERENCE TO RELATED APPLICATION

This application claims the benefit of U.S. Patent Application No. 61/474,631, filed Apr. 12, 2011, incorporated herein by reference in its entirety.

### STATEMENT OF GOVERNMENT LICENSE RIGHTS

This invention was made with Government support under DGE-0718124 awarded by the National Science Foundation Graduate Research Fellowship. The Government has certain rights in the invention.

### BACKGROUND OF THE INVENTION

In 2012, the American Cancer Society estimated that 22,910 malignant tumors of the brain or spinal cord will be diagnosed in the United States. Approximately 13,700 (59.8%) of these tumors will prove fatal. Current treatments encompass a variety of methods from surgical excision to radiation therapy and chemotherapy. Despite decades of research, survival in patients with malignant gliomas remains bleak with median survival approximating 1 year despite maximal therapy. Part of the reason that gliomas and other malignant tumors are so treatment resistant is that they evolve resistance to chemotherapeutics and radiation and can escape treatment because of the blood brain barrier. Thus, new treatments are required that deliver multi-drug individualized therapy locally to the site of the tumor. This has been attempted previously with limited success; GLIADEL® wafers (Eisai Inc.) are chemotherapy wafers that are applied locally to the surgical resection cavity. These wafers degrade over a period of 2-3 weeks, releasing chemotherapeutics to remaining tumor cells locally. However, the wafers have limited surface contact with the brain tissue and only release a single drug against which a tumor can develop resistance.

A need exists for a topical, slow release, multi-drug delivery system to be applied post-surgically to individuate therapy and increase the patient's life expectancy. The present invention seeks to fulfill this need and provides further related advantages.

### SUMMARY OF THE INVENTION

The present invention provides compositions and methods for delivering a therapeutic agent to a biological tissue.

In one aspect, the invention provides a composition that includes a temperature-responsive polymer and one or more degradable microspheres, each comprising a therapeutic agent.

In one embodiment, the composition comprises:

(a) a degradable microsphere having a degradation rate and comprising a first therapeutic agent; and

(b) a temperature-responsive polymer.

In another embodiment, the composition comprises:

(a) a first microsphere having a first degradation rate and comprising a first therapeutic agent;

(b) a second microsphere having a second degradation rate and comprising a second therapeutic agent, wherein the first and second degradation rates are different; and

(c) a temperature-responsive polymer.

For the above compositions, in certain embodiments, the first and second therapeutic agents are the same, and in other embodiments, the first and second therapeutic agents are different.

In one embodiment, the composition further comprises a third microsphere having a third degradation rate and comprising a third therapeutic agent, wherein the third degradation rate is different from the first and second degradation rates. For this composition, in certain embodiments, the first, second, and third therapeutic agents are the same, and in other embodiments, the first, second, and third therapeutic agents are different.

Representative microspheres useful in the compositions include poly(lactic acid), poly(ε-caprolactone), and poly(lactic-co-glycolic acid) microspheres.

Representative therapeutic agents useful in the compositions include chemotherapeutic agents and antibiotics.

Suitable temperature-responsive polymers useful in the compositions have a lower critical solution temperature from about 28 to about 35° C. In certain embodiments, the temperature-responsive polymer becomes adherent to biological tissue at a temperature above 32° C. Representative temperature-responsive polymers include degradable polymers, such as a degradable poly(N-isopropylacrylamide).

In certain embodiments, the compositions further include a pharmaceutically acceptable carrier.

In certain embodiments, the compositions are in the form of a suspension suitable for spraying onto a site of interest, such as biological tissue.

In certain embodiments, the compositions are in the form of a gel conformable to the contour of a biological tissue surface.

In another aspect, the invention provides a method for delivering a therapeutic agent to a site of interest. In the method, the composition is applied to a biological tissue and forms a gel that adheres to the biological tissue. Degradation of the microspheres adhered to the biological tissue releases the therapeutic agents to the tissue over time.

In one embodiment, the method comprises contacting the site with a composition of the invention. In one embodiment, the site is a biological tissue. Suitable sites include cancerous tissue, such as the surgical site after cancerous tissue resection.

In one embodiment, contacting the site with the composition comprises spraying the composition onto the site.

In another aspect of the invention, a method for treating a brain cancer is provided. In the method, brain tissue is contacted with a composition of the invention. In one embodiment, the brain tissue is a surgical site after cancerous tissue resection. In one embodiment, contacting brain tissue with the composition comprises spraying the composition onto the tissue.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a scanning electron microscope (SEM) micrograph of rhodamine B encapsulated poly(lactic acid) (PLA) microspheres (2800 rpm) at 313× magnification.

FIG. 2 is a SEM micrograph of rhodamine B encapsulated poly(ε-caprolactone) (PCL) microspheres (3240 rpm) at 313× magnification.

FIG. 3 is a SEM micrograph of rhodamine B encapsulated poly(lactic-co-glycolic acid) (PLGA) microspheres (2800 rpm) at 570× magnification.

FIG. 4 is a SEM micrograph of gefitinib encapsulated PLGA microspheres (double emulsion, 2000 rpm) at 1291× magnification.

FIG. 5 compares encapsulation efficiencies of rhodamine B using different polymeric microsphere types and formation parameters (double emulsion). Unless otherwise noted, rhodamine B was added at 1 mg/mL to the inner water phase.

FIG. 6 compares release profiles of rhodamine B from PLGA microspheres in PBS at 37° C.: diameter A, 42.2±15.4 μm (2000 rpm) and diameter B, 22.5±7.8 μm(2800 rpm).

FIG. 7 is a schematic illustration of the synthesis of a degradable linear poly(N-isopropylacrylamide) (polyNIPAM or PNIPAM).

FIG. 8 compares the transmittance of polyNIPAM solutions with different Mw polyNIPAM (10K, 20K, 40K) as a function of temperature. LCST was determined from the transmittance at 500 nm measured against temperature using an UV-Vis spectrophotometer coupled with a temperature controller. Sample concentration: 5% wt/v; λ=500 nm; path length=1 cm; heating rate about 1° C./min.

FIG. 9A-9C are images of brain tissue after four spray sets of polyNIPAM and rhodamine B encapsulated PLGA microspheres, 30 mg of rhodamine B encapsulated PLGA microspheres per 5 mL of PNIPAM (2.5% W_{PNIPAM}/V_{PBS}): light microscopy (9A), fluorescence microscopy (9B), and magnified fluorescence microscopy (9C).

### DETAILED DESCRIPTION OF THE INVENTION

The present invention provides compositions and methods for delivering a therapeutic agent to a biological tissue. The composition includes a temperature-responsive polymer and one or more degradable microspheres, each comprising a therapeutic agent. In the method, the composition is applied to a biological tissue and forms a gel that adheres to the biological tissue. Degradation of the microspheres adhered to the biological tissue releases the therapeutic agents to the tissue over time.

The compositions and methods provide topical, localized, and controlled delivery of therapeutic agents to a site of interest. Sites of interest that benefit from the delivery of therapeutic agents by the compositions and methods of the invention include the cancer sites such as the site of cancerous tumor resection. For these sites, the therapeutic agents include chemotherapeutic agents.

The compositions and methods are particularly useful for localized treatment of cancer in which the composition is applied to the surgical site after cancerous tissue resection. The therapeutic agents are released from the microspheres to the local tissues to treat residual cancerous tissue that may remain after surgery.

In one aspect, the invention provides a composition comprising a temperature-responsive polymer and one or more degradable microspheres, each comprising a therapeutic agent.

In one embodiment, the composition comprises:

(a) a first microsphere having a first degradation rate and comprising a first therapeutic agent; and

(b) a temperature-responsive polymer.

In another embodiment, the composition comprises:

(a) a first microsphere having a first degradation rate and comprising a first therapeutic agent;

(b) a second microsphere having a second degradation rate and comprising a second therapeutic agent,

wherein the first and second degradation rates are different, and

wherein the first and second therapeutic agents are different; and

(c) a temperature-responsive polymer.

In a further embodiment, the composition further includes a third microsphere having a third degradation rate and comprising a third therapeutic agent, wherein the third degradation rate is different from the first and second degradation rates, and wherein the third therapeutic agent is different from the first and second therapeutic agents.

Compositions including more than three types of microspheres are within the scope of the invention.

The embodiments of the compositions noted above refer to a “first microsphere,” a “second microsphere,” and a “third microsphere.” It will be appreciated that the each of these microspheres represents a microsphere type (e.g., a microsphere having a particular degradation rate) and that the composition includes a plurality of each type of the recited microsphere.

In the embodiments noted above, the first and second therapeutic agents, and the first, second, and third therapeutic agents, are different. However, in other embodiments, the first and second therapeutic agents, and the first, second, and third therapeutic agents, are the same.

For embodiments that include three or more types of microspheres, it will be appreciated that the different types of microspheres can include combinations of the same or different therapeutic agents (e.g., first and third microsphere types include the same agent and the second microsphere type includes a different agent).

For the purpose of delivery to a site of interest, the compositions of the invention include a carrier or diluent. Suitable carriers and diluents include pharmaceutically acceptable aqueous carriers and diluents. Representative carriers and diluents include phosphate buffered saline (PBS, e.g., buffered at physiological pH) and deionized water.

Suitable microspheres useful in the present invention include polymeric microspheres that are degradable in vivo. Through their biodegradation, the microspheres release their encapsulated therapeutic agent over time. Because each microsphere type degrades at a different rate, each microsphere delivers its encapsulated therapeutic agent to the site of interest at a different rate or at a time.

For a composition that includes two microspheres, in one embodiment, the first microsphere degrades and delivers substantially all of its encapsulated therapeutic agent (i.e., first therapeutic agent) before the second microsphere begins to degrade and thereby delivers its encapsulated therapeutic agent (i.e., second therapeutic agent) only after delivery of the first therapeutic agent. Advantages of this mode of delivery include increasing therapeutic efficiency in situations where therapeutic efficiency is decreased due to the development of drug resistance.

The degradation profiles of the microspheres (e.g., first, second, or third) need not be non-overlapping. The degradation of the microspheres (e.g., first, second, or third) can occur such that more than one therapeutic agent is delivered at a given time.

Representative microspheres useful in the composition and methods include poly(lactic acid), poly(ε-caprolactone), and poly(lactic-co-glycolic acid) microspheres. For poly(lactic-co-glycolic acid), the ratio of lactic acid and glycolic acid comonomers can be varied. In one embodiment, the ratio is 1:1.

In general, PLGA microspheres degrade more rapidly than PLA microspheres, which degrade more rapidly than PCL microspheres. Degradation rate can be modified by modifying microsphere hydrophilicity/hydrophobicity, sphere morphology and size, as well as polymer molecular weight.

Other suitable microspheres can be prepared from poly(glycolic acid), poly(methylidene malonate 2.1.2) (PMM 2.1.2), poly(3-hydroxybutyrate) (PHB), poly(3-hydroxybutyrateco-3-hydroxyvalerate), (P(HBco-HV)), polyanhydrides; aliphatic polycarbonates, polysaccharides (e.g., dextran, cellulose), chitosans, and proteins (e.g., collagen, fibrin, gelatin, albumin).

The microspheres are present in the composition in an amount ranging from about 0.01 to about 50 percent by weight based on the total weight of the composition. In one embodiment, the microspheres are present in the composition in about 0.05 to about 30 percent by weight based on the total weight of the composition. In one embodiment, the microspheres are present in the composition in about 0.1 to about 10 percent by weight based on the total weight of the composition. In another embodiment, the microspheres are present in the composition in about 0.1 to about 1 percent by weight based on the total weight of the composition.

Suitable therapeutic agents deliverable by the composition include any therapeutic agent that can be incorporated into a degradable microsphere. The choice of the therapeutic agent will depend on the nature of the site of interest receiving the composition. For sites of interest that include cancerous tissues, the therapeutic agent(s) are chemotherapeutic agents. For sites of interest that are infections, the therapeutic agent(s) are therapeutic agents useful in treating infections, such as antibiotics.

Representative therapeutic agents that are effectively delivered by the composition and in the methods of the invention include chemotherapeutic agents such as kinase inhibitors such as gefitinib, imatinib, SU11274, and CCI-779; alkylators such as temozolomide (TMZ) and irinotecan; anti-angiogenics such avastin (bevacizumab); differentiators such as bone morphogenetic protein (BMP); and bioenergetics such as 2-deoxyglucose, oxythiamine, 3-bromopyruvate, and alpha-aminocaproic acid. Other representative chemotherapeutic agents deliverable by the composition include paclitaxel, docetaxel, taxotere, camptothecin, carboplatin, BCNU, doxorubicin, and 6-fluorouracil.

The preparation and characterization of representative microspheres useful in the compositions and methods of the invention are described Example 1.

The composition of the invention includes a temperature-responsive polymer that serves as a matrix (e.g., host) to the microspheres. As noted above, by virtue of the temperature-responsive polymer, the composition to be administered is a liquid (e.g., liquid suspension of polymeric drug encapsulated microspheres) and on contact with a biological tissue at physiological temperature, the composition becomes a gel that adheres to the contours of the biological tissue at the site of application.

The temperature-responsive polymer has a lower critical solution temperature (LCST) from about 25 to about 40° C. In one embodiment, the LCST is from about 28 to about 35° C. In another embodiment, the LCST is from about 30 to about 32° C. The LCST of polyNIPAM is about 32° C. (sharp liquid-solid phase transition). In general, the LCST of polyNIPAM can be tuned by co-polymerization of NIPAM with hydrophobic or hydrophilic monomers. Co-polymerization with hydrophobic monomers decrease the LCST and co-polymerization with hydrophilic monomers and increase the LCST. The LCST can be shifted from 32° C. to the range between 20 to 60° C.

In one embodiment, the temperature-responsive polymer becomes adherent at a temperature above 32° C.

Suitable temperature-responsive polymers include polymers that are degradable in vivo. Representative temperature-responsive polymers include poly(N-isopropylacrylamide)s and polymers that include N-isopropylacrylamide repeating units.

The preparation and characterization of a representative temperature-sensitive polymer useful in the compositions and methods of the invention is described in Example 2 and illustrated in FIG. 7. The LSCT for the degradable polyNIPAMs described herein with Mn (theoretical) of 10, 20, and 40K is 30.2, 30.4 and 30.8° C., respectively.

The temperature-sensitive polymer is present in the composition in an amount ranging from about 0.01 to about 50 percent by weight based on the total weight of the composition. In one embodiment, the polymer is present in the composition in about 0.05 to about 30 percent by weight based on the total weight of the composition. In one embodiment, the polymer is present in the composition in about 0.1 to about 10 percent by weight based on the total weight of the composition. In another embodiment, the polymer is present in the composition in about 2 to about 5 percent by weight based on the total weight of the composition. In one embodiment that includes phosphate buffered saline as diluent, the composition includes 2.4% w/w polymer based on the total weight of the composition and 2.5% w/v polymer based on the volume of phosphate buffered saline.

As noted above, in one embodiment, the composition of the invention is in the form of a suspension suitable for spraying onto a biological tissue surface. In another embodiment, the composition is in the form of a gel that is conformable to the contour of a biological tissue surface.

The preparation and characterization of a representative drug delivery system of the invention is described in Example 3.

In another aspect of the invention, a method for delivering a therapeutic agent to a site of interest is provided. In the method, a composition comprising a temperature-responsive polymer and one or more degradable microspheres, each comprising a therapeutic agent (same or different), is contacted with the site. In one embodiment, the method comprises delivering a first therapeutic agent to a site by contacting the site with a composition of the invention described herein. In another embodiment, the method comprises delivering a first and a second therapeutic agent to a site by contacting the site with a composition of the invention described herein. In a further embodiment, the method comprises delivering a first, a second, and a third therapeutic agent to a site by contacting the site with a composition of the invention described herein.

The site to which the composition applied is a biological tissue. The biological tissue can be any tissue to be targeted for therapeutic agent delivery. In one embodiment, the site is the site of cancerous tissue and the therapeutic agent delivered is a chemotherapeutic agent. In one embodiment, the biological tissue is a surgical site after cancerous tissue resection (e.g., post-surgical brain tissue). In another embodiment, the site is the site of infection and the therapeutic agent delivered is an antibiotic.

In the method, the composition contacts the site of interest by applying the composition to the site. In one embodiment, contacting the site with the composition comprises spraying the composition onto the site. It will be appreciated that the composition can be applied by any technique suitable for topical administration of a liquid composition.

In one specific embodiment, the invention provides a method for treating a brain cancer, comprising contacting brain tissue with a composition of the invention. In this embodiment, the brain tissue is a post-surgical site (i.e., after cancerous tissue resection). In one embodiment, the composition is applied to the surgical site by spraying the composition onto the site.

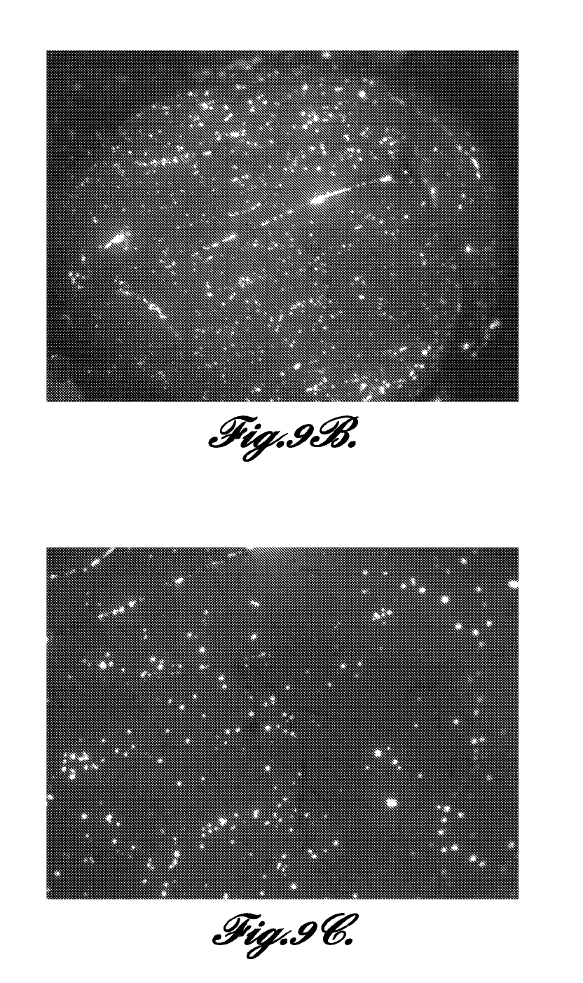

Spraying effectively delivered the drug delivery system and produced a uniform distribution that adhered to the tissue due to the elevated temperature. FIGS. 9A-9C are images showing the results of spraying PNIPAM suspending rhodamine B encapsulated PLGA microspheres. FIG. 9A is a light microscope image of brain tissue. FIG. 9B is a fluorescence microscope image of the brain tissue after application of the composition. FIG. 9C is a magnified fluorescence microscope image. Referring to FIGS. 9A-9C, it is clear that the loaded microspheres were successfully delivered.

As noted above, in one aspect, the present invention provides a drug delivery system that is useful for localized brain tumor therapy. In one embodiment, the system includes poly(lactic acid) (PLA), poly(lactic-co-glycolic acid) (PLGA), and poly(ε-caprolactone) (PCL) microspheres, each including a different therapeutic agent, suspended in a biodegradable poly(N-isopropylacrylamide) (PNIPAM) matrix. At room temperature, PNIPAM is capable of suspending drug encapsulated microspheres that can be sprayed on the post-surgical site. The 37° C. temperature of this site would pass PNIPAM through its lower critical solution temperature, causing the polymer matrix to solidify on the surface of the brain, providing intimate contact with the remaining tumor cells. Over time, PLGA, PLA, and then PCL degrade releasing multiple chemotherapeutics at different rates directly to the tumor remnants to inhibit cancerous re-growth.

The following examples are provided for the purpose of illustrating, not limiting, the invention.

### EXAMPLES

### Example 1

### The Preparation and Characterization of Representative Microspheres

In this example, the preparation and characterization of representative microspheres of the invention is described.

Microsphere Preparation.

PLGA, PLA, and PCL microspheres were produced by either an oil/water single emulsion or a water/oil/water double emulsion, solvent evaporation technique known to those of skill in the art. For the double emulsion, 1.75 g of the polymer was dissolved in 35 mL of dichloromethane (O) and 0.5 g of poly(vinyl alcohol) was dissolved in 50 mL of deionized water (W2). The 0 solution was homogenized with 1.5 mL deionized water (W1) at 6,000 rpm for two minutes using an Arrow 6000 electric stirrer. Then, 7 mL of this solution was added to the W2 solution and homogenized for one minute at a given speed. The W1/O/W2 solution sat overnight and then was stirred for two hours for solvent evaporation. The solution was centrifuged at 3,000 g and 4° C. for 15 minutes with three water rinse cycles before being passed through two Whatman No. 4 filters. Drug encapsulated spheres were made by adding the drug to the W1 phase, normally at 1 mg/mL unless otherwise noted. For the single emulsion, the W1 phase was not used and the drugs were added straight to the oil phase. For both emulsion system, 3% poly(vinyl alcohol) (W/V) was sometimes used and is noted accordingly. All emulsion steps used a five blade, circular impeller.

Rhodamine B (RB) is a hydrophilic, fluorescent dye that was chosen as a model drug for the target chemotherapeutic, gefitinib, because of its similar molecular weight and ring structure. Fluorescein (F) is a hydrophobic, fluorescent dye that was chosen as a model drug for hydrophobic chemotherapeutic drugs.

Unless otherwise stated, all results are from the water in oil in water, double emulsion formation technique.

Microsphere Characterization.

Microsphere shape, size, and fluorescent capabilities were determined using a Nikon E800 Upright Microscope. Microsphere morphology was determined using a FEI Sirion XL30 scanning electron microscope. Microsphere size was determined using a Horiba LA950 laser diffraction particle size distribution analyzer.

TABLE 1
Microsphere Particle Size (Horiba LA950).

| | | Average | |
|---|---|---|---|
| Polymer | Conditions | (μm) | Standard Deviation (μm) |
| PCL | 2800 rpm | 48.6 | 16.6 |
| PCL | 2800 rpm, 1 mg/mL RB | 52.1 | 19.9 |
| PCL | 3240 rpm | 44.0 | 17.7 |
| PCL | 3240 rpm, 1 mg/mL RB | 50.7 | 19.5 |
| PLA | 2800 rpm | 37.2 | 13.1 |
| PLA | 2800 rpm, 1 mg/mL RB | 37.0 | 10.8 |
| PLA | 3560 rpm | 29.2 | 10.1 |
| PLA | 3560, 1 mg/mL RB | 27.4 | 9.9 |
| PLGA | 2000 rpm | 35.2 | 13.8 |
| PLGA | 200 rpm, 1 mg/mL RB | 42.2 | 15.4 |
| PLGA | 2800 rpm | 24.6 | 8.9 |
| PLGA | 2800 rpm, 1 mg/mL RB | 22.5 | 7.8 |

PLA Microspheres.

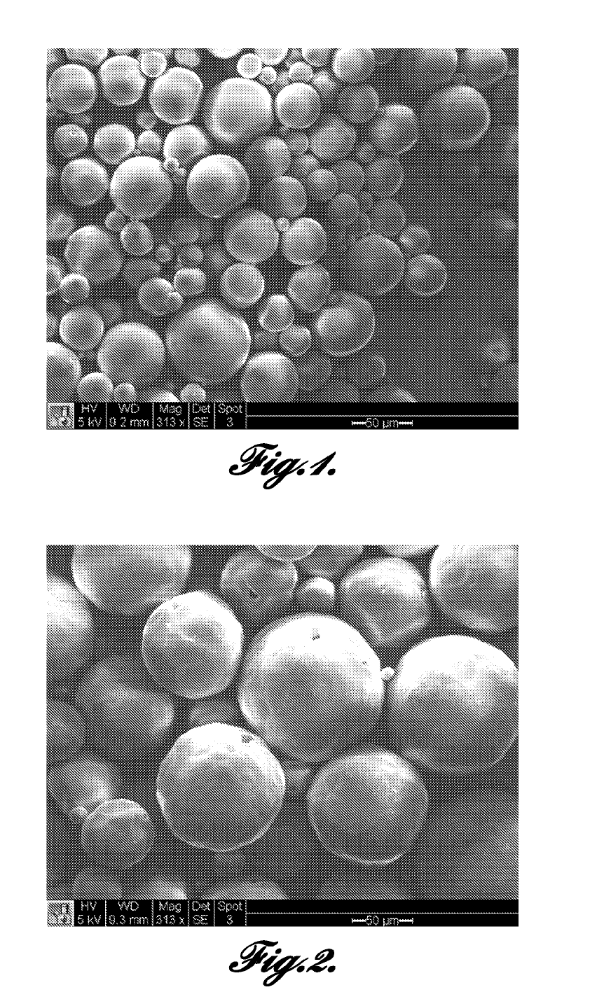

PLA sphere formation was subjected to the two speeds of 2800 and 3560 rpm. The slower speed resulted in larger spheres 37.2±13.1 μm in diameter and the faster speed resulted in smaller spheres 29.2±10.1 μm in diameter. Scanning electron microscopy (SEM) showed that the surface morphology was smooth and nonporous. Rhodamine B encapsulation resulted in sizes 37.0±10.8 μm for 2800 rpm and 27.4±9.9 μm for 3560 rpm. The dye was present when viewed with fluorescent microscopy and the sphere morphology remained unaffected by the dye when characterized by SEM (FIG. 1). Surface localized rhodamine B crystals were not seen with SEM, indicating that the dye was encapsulated inside the microsphere. The small, outward facing pits on the surface seen with the close up views of the SEM are most likely due to solvent evaporation during microsphere formation.

PCL Microspheres.

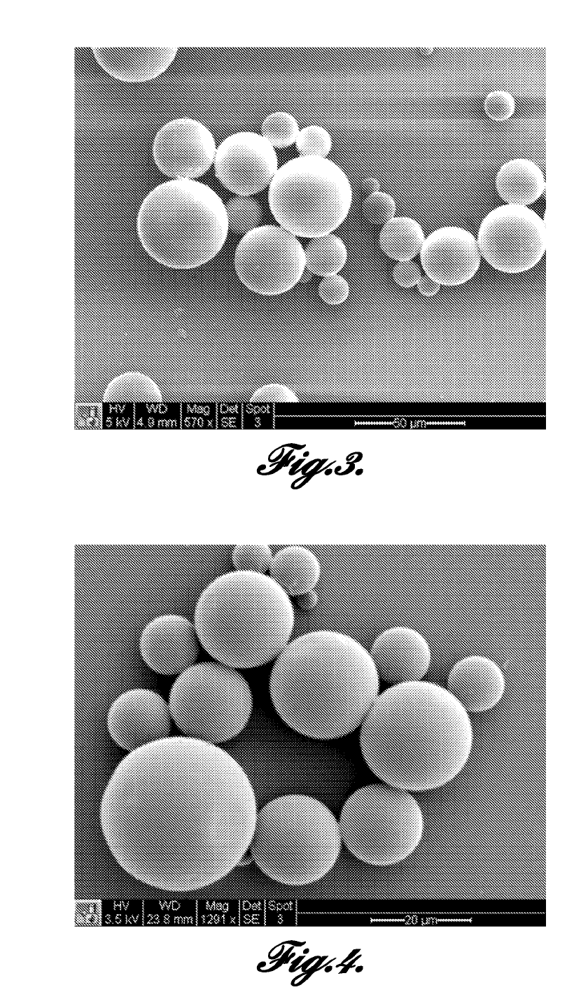

PCL sphere formation was subjected to the two speeds of 2800 and 3240 rpm. The faster speed resulted in spheres 44.0±17.7 μm in diameter while the slower speed had spheres 48.6±16.6 μm. When viewed with SEM, the majority of the surfaces were smooth, non-porous, and spherical. Rhodamine B encapsulation was then tested, with similar sizes resulting: 52.1±19.9 μm for 2800 rpm and 50.7±19.5 μm for 3240 rpm. When viewed with fluorescent microscopy, spheres were successfully loaded with rhodamine B. SEM showed that PCL microspheres were also unaffected by the dye, remaining smooth and spherical with no surface localized dye crystals seen (FIG. 2).

PLGA Microspheres.

PLGA sphere formation was subjected to the two speeds of 2000 and 2800 rpm. The slower speed resulted in an average sphere diameter of 35.2±13.8 μm while the faster speed had an average sphere diameter of 24.6±8.9 μm. Utilizing SEM, surface morphology was determined to be smooth and nonporous. Rhodamine B was encapsulated at the same speeds, resulting in sphere diameters of 42.2±15.4 μm for 2000 rpm and 22.5±7.8 μm for 2800 rpm. The dye was present when viewed with fluorescent microscopy and the sphere morphology remained unaffected by the dye when characterized by SEM (FIG. 3).

PLGA/Gefitinib Microspheres.

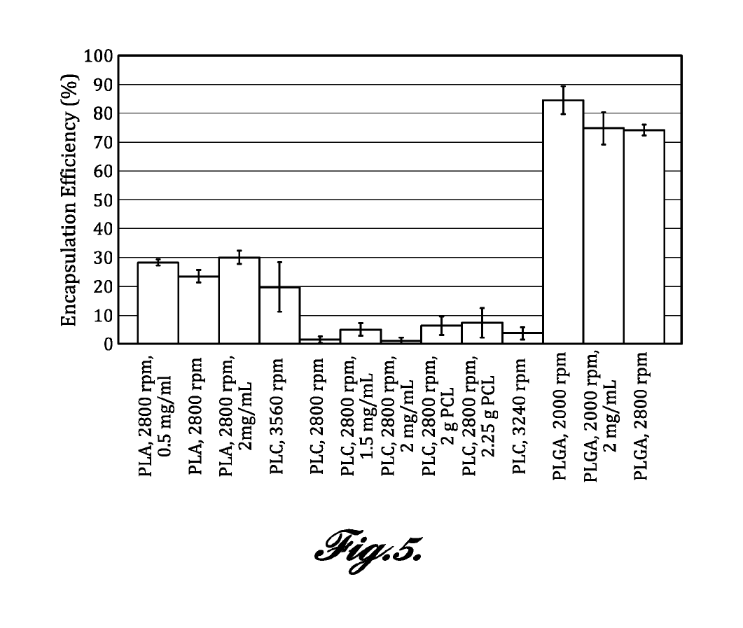

Gefitinib is a chemotherapeutic that prevents the uncontrolled cell proliferation that is common in cancerous tumors. Gefitinib also happens to have a fluorescent emission that can be seen using microscopy. PLGA was chosen as a representative carrier for gefitinib. As determined by fluorescence microscopy, gefitinib was successfully encapsulated. Without the presence of gefitinib, PLGA microspheres have no noticeable emission at the wavelength studied. SEM of the PLGA encapsulated microspheres showed a surface morphology that was smooth and nonporous, without the presence of any surface localized drug crystals (FIG. 4). A single, oil in water emulsion was also used to make gefitinib encapsulated PLGA microspheres. Similar results were seen as that for the double emulsion microspheres. Gefitinib was present as demonstrated with fluorescent microscopy and the surface morphology was smooth and nonporous, without the presence of any surface localized drug crystals.

Hydrophobic Dye Encapsulation.

Fluorescein, a hydrophobic, fluorescent dye, was used to model the encapsulation of a hydrophobic drug. PLA, PCL, and PLGA were capable of forming microspheres encapsulating fluorescein that were readily viewed by fluorescent microscopy.

Encapsulation Efficiency.

Encapsulation efficiency is a measurement of how well the microsphere encapsulated the amount of drug that is initially added to the formation steps:

EE=wt drug encapsulated/wt theoretical drug encapsulated

As can be seen in FIG. 5, the greatest contributing factor to the encapsulation efficiency of rhodamine B is the polymer type. The most hydrophilic polymer, PLGA, resulted in the highest encapsulation for the hydrophilic dye (84.6±4.8% at 2000 rpm). PLA had the second highest encapsulation at 23.5±2.2% for 2800 rpm followed by a small percentage for PCL, a hydrophobic polymer, at 3.7±2.1% for 3240 rpm. Increasing the initial drug loading and polymer concentration did not result in a significant change for PCL encapsulation efficiency. However, a modest increase of about 7% was seen when the rhodamine B was loaded at 2 mg/mL for PLA.

In Vitro Release of Rhodamine B from PLGA Microspheres.

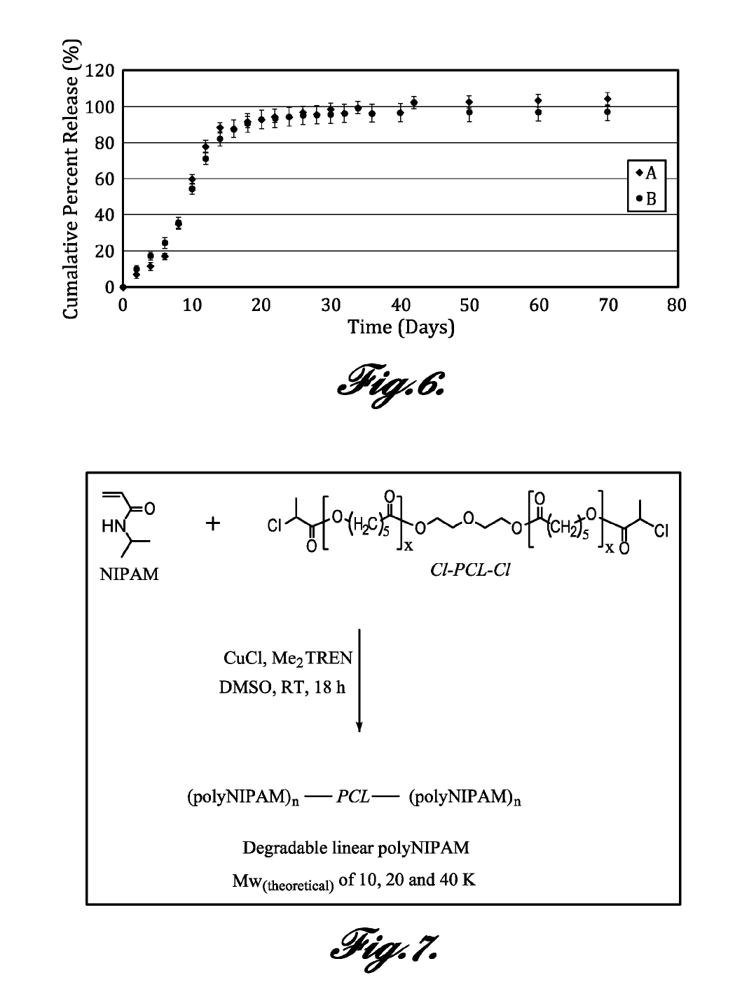

The in vitro release profile of rhodamine B from PLGA microspheres is shown in FIG. 6. As can be seen, two different regimes of controlled release can be identified. Up until day 8, there is a moderate, continuous release that can be attributed to diffusion. After day 8, there is a significant increase in the slope of the release curve, indicating degradation of the polymer that results in a larger release rate. A small difference between the initial sizes of the two polymer sets can be noted, with the smaller microsphere (higher impeller speed setting) releasing faster during the diffusion period due to its higher surface area to volume ratio. This shows a degree of tunability for a desired release profile.

### Example 2

### The Preparation and Characterization of a Representative Biodegradable Host Polymer

In this example, the preparation and characterization of a representative host polymer useful for delivering loaded polymeric microspheres is described.

Difunctional Macroinitiator (Cl-PCL-Cl).

The preparation of a macroinitiator for preparing a degradable host polymer useful in the drug delivery system of the invention is illustrated schematically below.

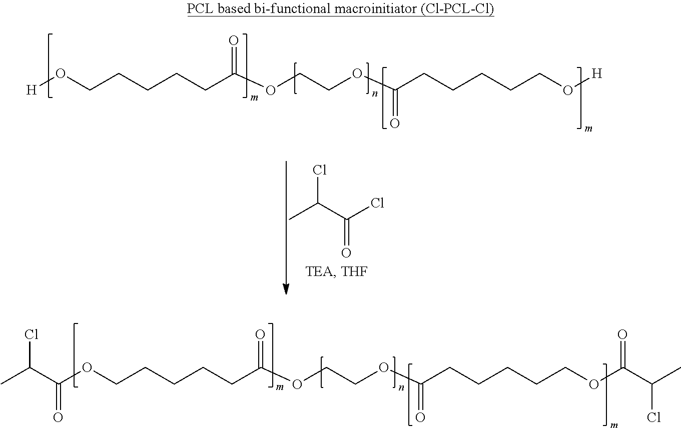

Polycaprolactonediol (Mn about 530, 5 g, 9.4×10⁻³ mol) and triethylamine (TEA) (6.5 mL, 0.047 mol) were dissolved in anhydrous tetrahydrofuran (THF) (150 mL) and the solution was cooled to 4° C. Chloropropionyl chloride (2.3 mL, 0.024 mol) was added dropwise under nitrogen atmosphere and the reaction mixture was stirred at room temperature (RT) for 18 h. The white precipitate was removed by filtration and the solvent was evaporated. The material was then dissolved in dichloromethane and washed with saturated NaHCO₃, 1N HCl, and H₂O. The organic phase was dried over MgSO₄, filtered and evaporated to produce yellow oil (yield 92%). ¹H NMR (CDCl₃): δ 1.30 (m, —COOCH₂CH₂CH₂CH₂CH₂COO—), 1.52 (m, —COOCH₂CH₂CH₂CH₂CH₂COO— and ClCH(CH₃)COO—), 2.35 (m, —COOCH₂CH₂CH₂CH₂CH₂COOCH₂CH₂O—), 3.75 (m, COO—CH₂CH₂—O—CH₂CH₂—OOC—), 4.0-4.6 (m, —COOCH₂CH₂CH₂CH₂CH₂COOCH₂CH₂O— and ClCH(CH₃)COO—).

Tris[2-(dimethylamino)ethyl]amine (Me₆TREN).

The preparation of Me₆TREN is illustrated below.

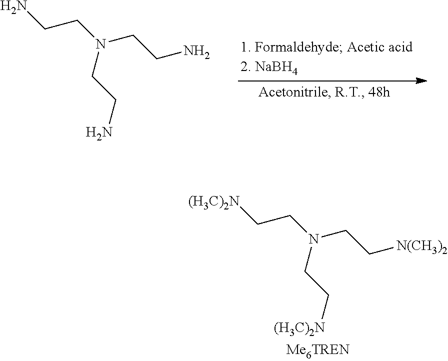

Tris(2-aminoethyl) amine (TREN) (3 mL, 19.9×10⁻³ mol) and acetic acid were dissolved in 600 mL of acetonitrile. Aqueous formaldehyde (37% wt, 49 mL, 660×10⁻³ mol) was added to the solution and the mixture was stirred at room temperature for 1 h. The reaction mixture was placed in an ice bath and sodium borohydride (10 g, 13.4×10⁻³ mol) was slowly and carefully added. After being stirred for 48 h at RT, the solvent was evaporated to obtain a yellow solid. 3M NaOH solution was added to dissolve the solid (final pH 11) and the product was extracted three times with dichloromethane. The organic phase was dried over MgSO₄, filtered and evaporated to obtain a yellow oil (yield 88%). ¹H NMR (CDCl₃): δ 2.2 (s, 18H, —CH₃), 2.32 (m, 6H, —NCH₂CH₂N(CH₃)₂), 2.60 (m, 6H, —NCH₂CH₂N(CH₃)₂).

Degradable Linear PolyNIPAM.

Atom transfer radical polymerization (ATRP) of NIPAM in presence difunctional PCL-based initiator leads to formation of a linear polyNIPAM with degradable sites in the backbone (FIG. 7). ATRP also allows governing polyNIPAM Mw, which is useful for tuning physical properties of the hydrogel toward desired applications.

In a typical procedure to obtain degradable linear polyNIPAM-20 based on backbone with target MW of 20K, NIPAM (1 g, 0.00884 mol), Cl-PCL-Cl (36 mg, 5×10⁻⁵ mol) were dissolved in dimethyl sulfoxide (DMSO) (1 mL) and the solution was purged with argon for 1 h. Then in the inert atmosphere CuCl (50 mg, 0.0005 mol) was dissolved in the purged solution following by addition of Me₆TREN (115 μl, 0.0005 mol). The reaction mixture stirred polymerized in inert atmosphere at RT for 18 h. After polymerization the material was purified by dialysis against water, lyophilized, dissolved in chloroform, precipitated from cold diethyl ether and dried under vacuum to obtain linear polyNIPAM-20 as a white powder. Degradable linear polyNIPAM-10 and polyNIPAM-40 with backbone target Mw of 10 and 40K, respectively, were synthesized by the same procedure at appropriate [NIPAM]/[Initiator] molar ratios (see Table 2). In these preparations, the molar ratio between [Initiator]/[CuCl]/[Me₆TREN] was constant at 1:10:10.

Because ATRP is controlled polymerization, polyNIPAM chains grow at the same rate from each end of difunctional initiator (i.e., Mw of the degradation products are half of the Mw of parent polymer).

Linear degradable polyNIPAM with theoretical Mw of 10, 20 and 40K was synthesized as described above. Table 2 summarizes Mw and PDI of the linear polymers and their degradation products.

TABLE 2
Mw of the degradable linear polyNIPAM and it degradation products.

| [NIPAM] | Before Degradation | | | After Degradation | |
|---|---|---|---|---|---|
| [Initiator], | Mn | Mn | PDI | Mn | PDI |
| (mol/mol) | (theory) | (GPC) | Mw/Mn (GPC) | (GPC) | Mw/Mn (GPC) |
| 88 | 10 000 | 11 710 | 1.38 | 5 430 | 1.65 |
| 177 | 20 000 | 22 954 | 1.14 | 13 807 | 1.16 |
| 353 | 40 000 | 35 324 | 1.09 | 18 937 | 1.17 |

Mw of the linear polymers before degradation demonstrates controlled polymerization with low polydispersity. Mw of the degradation products are about half of the Mw of the parent polyNIPAM with slight increase in PDI. This data supports polyNIPAM chain growth at the same rate from each end of the difunctional macroinitiator.

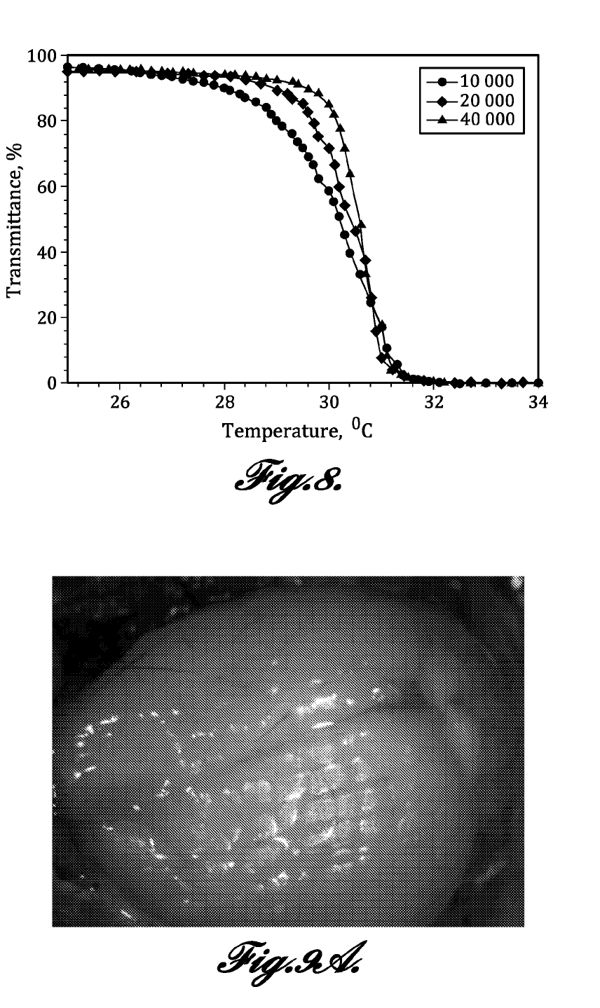

Aqueous solutions of degradable polyNIPAM with theoretical Mw of 10, 20, and 40K demonstrate lower critical solution temperature (LSCT) around 30° C., which is below body temperature (FIG. 8). This allows polyNIPAM solution to form solid hydrogel at body temperature.

### Example 3

### The Preparation and Characterization of a Representative Drug Delivery System

In this example, the preparation and characterization of a representative drug delivery system of the invention is described.

PNIPAM Clot.

A thermoresponsive PNIPAM clot was prepared to test the release of rhodamine B encapsulated PLGA microspheres. PNIPAM becomes a gel-like disc when heated about 32° C. A 1 mL of “blank” PNIPAM (2.5% W_{PNIPAM}/V_{PBS}) without any microspheres present was prepared as a control. Rhodamine B encapsulated PLGA microspheres in a PNIPAM clot was formed from 1 mL of PNIPAM solution (2.5% W_{PNIPAM}/V_{PBS}) containing 15 mg of dispersed microspheres. The PNIPAM clot was capable of retaining the microspheres once it collapsed due to a temperature increase.

Spraying Rhodamine B Microspheres Suspended in PNIPAM on Heated Rat Brain Tissue.

PNIPAM suspending rhodamine B encapsulated PLGA microspheres was sprayed on a heated rat brain (in vitro experiment) Spraying effectively delivered the drug delivery system and produced a uniform distribution that adhered to the tissue due to the elevated temperature. FIGS. 9A-9C are microscopic images showing the results of four pump sprays of PNIPAM suspending rhodamine B encapsulated PLGA microspheres. The conditions of the system are 30 mg of rhodamine B encapsulated PLGA microspheres per 5 mL of PNIPAM (2.5% W_{PNIPAM}/V_{PBS}).

Referring to FIGS. 9A-9C, the loaded microspheres were successfully delivered. The concentration of microspheres is spray dependent; a higher density of microspheres present when a total of ten spray pumps were used.

While illustrative embodiments have been illustrated and described, it will be appreciated that various changes can be made therein without departing from the spirit and scope of the invention.

1. A composition, comprising:
(a) a first microsphere having a first degradation rate and comprising a first therapeutic agent; and
(b) a temperature-responsive polymer.

2. The composition of claim 1 further comprising a second microsphere having a second degradation rate and comprising a second therapeutic agent, wherein the first and second degradation rates are different.

3. The composition of claim 2, wherein the first and second therapeutic agents are the same.

4. The composition of claim 2, wherein the first and second therapeutic agents are different.

5. The composition of claim 2 further comprising a third microsphere having a third degradation rate and comprising a third therapeutic agent, wherein the third degradation rate is different from the first and second degradation rates.

6. The composition of claim 5, wherein the first, second, and third therapeutic agents are the same.

7. The composition of claim 5, wherein the first, second, and third therapeutic agents are different.

8. The composition of claim 1, wherein the microspheres are selected from the group consisting of poly(lactic acid), poly(ε-caprolactone), and poly(lactic-co-glycolic acid) microspheres.

9. The composition of claim 1, wherein the therapeutic agent is a chemotherapeutic agent.

10. The composition of claim 1, wherein the temperature-responsive polymer has a lower critical solution temperature from about 28 to about 35° C.

11. The composition of claim 1, wherein the temperature-responsive polymer becomes adherent to biological tissue at a temperature above 32° C.

12. The composition of claim 1, wherein the temperature-responsive polymer is a degradable polymer.

13. The composition of claim 1, wherein the temperature-responsive is a degradable poly(N-isopropylacrylamide).

14. The composition of claim 1 further comprising a pharmaceutically acceptable carrier.

15. The composition of claim 1 in the form of a suspension suitable for spraying.

16. The composition of claim 1 in the form of a gel conformable to the contour of a biological tissue surface.

17. A method for delivering a therapeutic agent to a site, comprising contacting the site with the composition of claim 1.

18. The method of claim 17, wherein the site is a biological tissue.

19. The method of claim 17, wherein the site is a surgical site after cancerous tissue resection.

20. The method of claim 17, wherein contacting the site with the composition comprises spraying the composition onto the site.

21. A method for treating a brain cancer, comprising contacting brain tissue with a composition of claim 1.

22. The method of claim 21, wherein the brain tissue a surgical site after cancerous tissue resection.

23. The method of claim 21, wherein contacting brain tissue with the composition comprises spraying the composition onto the tissue.
